(19) United States
(12) Patent Application Publication
(10) Pub. No.: US 2025/0213782 A1
(43) Pub. Date: Jul. 3, 2025

(54) MEDICAL DEVICE HUB CONNECTION SYSTEM, METHOD, AND APPARATUS

(71) Applicants: BAXTER INTERNATIONAL INC., Deerfield, IL (US); BAXTER HEALTHCARE SA, Glattpark (Opfikon) (CH)

(72) Inventors: Peter BOJAN, Grayslake, IL (US); Gnana D. PRASUNA, Andhra Pradesh (IN); Siba Prasad SAHU, Bangalore (IN); David James SCHMIDT, Gurnee, IL (US); Subbaiah PATIBANDLA, Karnataka Bangalore (IN); Erik Michael THOMAS, Gurnee, IL (US); Niranjan MANJUNATH, Bangalore (IN); Yogesh MUNAVALLI, Karnatake Bangalore (IN); Jiri SLABY, Buffalo Grove, IL (US); Sridhar Arjun BELAKAWADI, Bangalore Karnataka (IN)

(21) Appl. No.: 19/005,098

(22) Filed: Dec. 30, 2024

(30) Foreign Application Priority Data
Dec. 28, 2023 (IN) 202341089496

(51) Int. Cl. A61M 5/142; G06F 1/16

(52) U.S. Cl. CPC A61M 5/142; G06F 1/1632; A61M 2209/086

(57) ABSTRACT

A medical device hub power management system, method, and apparatus are disclosed. An example infusion pump docking apparatus of a hub connectivity station includes a housing and a device connector assembly. The device connector assembly includes a mounting bracket fixedly connected to an interior surface of the housing forming a mounting cavity between the mounting bracket and the housing. The device connector assembly also includes a device connector to provide electrical connection between the docking apparatus and a medical device, where the device connector is positioned in the mounting cavity. The device connector assembly also includes a spring positioned in the mounting cavity between a flange surface of the mounting bracket and a rear surface of the device connector. The spring is deformable during connection of the medical device to the device connector to allow movement of the device connector within the mounting cavity.

### PRIORITY CLAIM

This application claims priority to and the benefit as a non-provisional application of Indian Provisional Patent Application No. 20/234,1089496, filed Dec. 28, 2023, the entire contents of which are hereby incorporated by reference and relied upon.

### BACKGROUND

Infusion pumps are medical devices that infuse a fluid into a patient's bloodstream or subcutaneously. Infusion therapies often use pumps to delivery nutrition in addition to certain medications such as pain medications, antibiotics, antiemetics, antifungals, antivirals, biologics, blood factors, chemotherapy drugs, corticosteroids, growth hormones, immunoglobulin replacement fluids, immunotherapy fluids, and inotropic heart medications. There are different types of infusion pumps that are optimized for delivering different types of fluids or medications. For instance, a syringe pump may be used for delivering low volumes of potent medications at relatively slow rates while large volume parenteral (“LVP”) pumps are used for delivering greater volumes of nutritional fluids. Other types of known infusion pumps include linear peristaltic pumps, patient-controlled analgesia (“PCA”) pumps, ambulatory pumps, and multi-channel pumps.

Some patient treatments may require the use of more than one infusion pump. For example, a first infusion pump may deliver saline while a second pump delivers a medication. Currently, clinicians can either connect individual pumps to a patient or use a multi-channel pump. When individual pumps are used, the pumps are often spread around a patient's hospital bed. The clinician has to move around the patient's bed to program each pump individually. In addition, the pumps consume a significant amount of space. When a multi-channel pump is used, a clinician only has to program one device and less space is used. However, multi-channel pumps generally only support one type of infusion pump, such as large volume pumps. If another pump is needed for a treatment, a clinician still has to add that infusion pump in addition to the multi-channel pump, thereby reducing the efficiencies of the multi-channel pump.

A need accordingly exists for an infusion pump hub that supports different types of infusion pumps.

### SUMMARY

An example system, method, and apparatus are disclosed for connectors configured to connect medical devices to a hub connectivity station. Unlike known multi-channel infusion pump systems with a fixed two, four, or six pumps, the hub connectivity station described herein is modular. In a base configuration, the hub connectivity station includes a single medical device (infusion pump) docking apparatus and a connectivity stage. As disclosed herein, the connectivity stage connects the hub to a hospital network. The docking apparatus includes two shelves for respectively receiving infusion pumps or other medical devices. In an embodiment, the shelves are configured to interchangeably accommodate different types of infusion pumps, such as syringe pumps, LVP pumps, PCA pumps, etc. based on which type of pump is needed for a particular treatment. Further, depending on the number of infusion pumps needed, additional docking apparatuses may be added in a stacked configuration. The hub connectivity station accordingly provides a compact and adaptable infusion management system that requires a relatively small footprint.

The example docking apparatus includes one or more connectors for mechanically, electrically, and/or communicatively coupling to medical devices, such as infusion pumps. The connector is configured to bend, translate, and/or rotate to enable a medical device to be more easily connected. Instead of having to perfectly line up a connector on a medical device to the connector of the docking apparatus disclosed herein, the example connector bends, flexes translates, and/or rotates to receive the medical device connector. This bending, flexing, translating, and/or rotating allows for some initial misalignment. However, as the medical device connector is pushed further into the docking apparatus connector, the docking apparatus connector aligns the medical device for connection to the docking apparatus. The example connector accordingly makes it easier to connect a medical device to a docking apparatus while facilitating a robust connection.

Aspects of the subject matter described herein may be useful alone or in combination with one or more other aspect described herein. Without limiting the foregoing description, in a first aspect of the present disclosure, an infusion pump docking apparatus includes a housing and a device connector assembly. The device connector assembly includes a mounting bracket fixedly connected to an interior surface of the housing forming a mounting cavity between the mounting bracket and the housing, a device connector to provide electrical connection between the docking apparatus and a medical device, the device connector positioned in the mounting cavity, and a spring positioned in the mounting cavity between a flange surface of the mounting bracket and a rear surface of the device connector, the spring deformable during connection of the medical device to the device connector to allow movement of the device connector within the mounting cavity.

In accordance with a second aspect of the present disclosure, which may be used in combination with the first aspect, the device connector assembly further comprises a front seal around the device connector, the front seal proximate an exterior surface of the housing.

In accordance with a third aspect of the present disclosure, which may be used in combination with any other aspect disclosed herein, the medical device is an infusion pump.

In accordance with a fourth aspect of the present disclosure, which may be used in combination with any other aspect disclosed herein, the device connector has freedom of motion in one or more directions.

In accordance with a fifth aspect of the present disclosure, which may be used in combination with any other aspect disclosed herein, the device connector moves from an initial position upon mounting of the medical device to the docking apparatus.

In accordance with a sixth aspect of the present disclosure, which may be used in combination with any other aspect disclosed herein, the interior surface of the housing proximate the device connector includes a housing tapered surface and the device connector includes a connector tapered surface.

In accordance with a seventh aspect of the present disclosure, which may be used in combination with any other aspect disclosed herein, the housing tapered surface and the connector tapered surface interact such that the device connector returns to the initial position after dismount of the medical device.

In accordance with an eighth aspect of the present disclosure, which may be used in combination with any other aspect disclosed herein, the device connector provides one or more of a controller area network connection or an Ethernet connection.

In accordance with a ninth aspect of the present disclosure, which may be used in combination with any other aspect disclosed herein, the device connector provides DC power to the medical device.

In accordance with a tenth aspect of the present disclosure, which may be used in combination with any other aspect disclosed herein, the device connector provides data routing between the medical device and the docking apparatus.

In accordance with an eleventh aspect of the present disclosure, which may be used in combination with any other aspect disclosed herein, the device connector assembly further includes one or more wires extending from the device connector.

In accordance with a twelfth aspect of the present disclosure, which may be used in combination with any other aspect disclosed herein, the device connector further includes a cylindrical protrusion extending from an exterior surface of the housing and a base extending from an interior surface of the housing.

In accordance with a thirteenth aspect of the present disclosure, which may be used in combination with any other aspect disclosed herein, the cylindrical protrusion of the device connector further includes a connection cavity extending inward from a front surface of the cylindrical protrusion.

In accordance with a fourteenth aspect of the present disclosure, which may be used in combination with any other aspect disclosed herein, the device connector further includes one or more pins extending outward from a rear surface of the connection cavity.

In accordance with a fifteenth aspect of the present disclosure, which may be used in combination with any other aspect disclosed herein, the movement of the device connector within the mounting cavity during connection of the medical device guides a connector of the medical device to the one or more pins to form the electrical connection.

In accordance with a sixteenth aspect of the present disclosure, which may be used in combination with any other aspect disclosed herein, an infusion pump docking apparatus of a hub connectivity station includes a housing, a first device connector assembly, and a second device connector assembly. The first device connector assembly includes a first mounting bracket fixedly connected to an interior surface of the housing forming a first mounting cavity between the first mounting bracket and the housing, a first device connector to provide electrical connection between the docking apparatus and a first medical device, the device connector positioned in the first mounting cavity, and a first spring positioned in the first mounting cavity between a flange surface of the first mounting bracket and a rear surface of the first device connector. The second device connector assembly includes a second mounting bracket fixedly connected to an interior surface of the housing forming a second mounting cavity between the second mounting bracket and the housing, a second device connector to provide electrical connection between the docking apparatus and a second medical device, the device connector positioned in the second mounting cavity, and a second spring positioned in the second mounting cavity between a flange surface of the second mounting bracket and a rear surface of the second device connector.

In accordance with a seventeenth aspect of the present disclosure, which may be used in combination with any other aspect disclosed herein, the first medical device is a first type of medical device and the second medical device is a second type of medical device.

In accordance with an eighteenth aspect of the present disclosure, which may be used in combination with any other aspect disclosed herein, an infusion pump docking apparatus of a hub connectivity station includes a housing, a device connector assembly, one or more couplings, and a shelf. The device connector assembly includes a mounting bracket fixedly connected to an interior surface of the housing forming a mounting cavity between the mounting bracket and the housing, a device connector to provide electrical connection between the docking apparatus and a medical device, the device connector positioned in the mounting cavity, and a spring positioned in the mounting cavity between a flange surface of the mounting bracket and a rear surface of the device connector. The one or more couplings provide mechanical coupling between the housing and the medical device. The shelf extends from the housing and supports the weight of the medical device when connected to the housing.

In accordance with a nineteenth aspect of the present disclosure, which may be used in combination with any other aspect disclosed herein, the shelf includes one or more recessed sections to guide the medical device into the one or more couplings.

In accordance with a twentieth aspect of the present disclosure, which may be used in combination with any other aspect disclosed herein, the apparatus further includes a release mechanism to decouple the one or more couplings from the medical devices.

In accordance with a twenty-first aspect of the present disclosure, any of the structure and functionality illustrated and described in connection with FIGS. 1 to 11 may be used in combination with any of the structure and functionality illustrated and described in connection with any of the other of FIGS. 1 to 11 and with any one or more of the preceding aspects.

In light of the present disclosure and the above aspects, it is therefore an advantage of the present disclosure to provide a device connector assembly on a hub connectivity station that utilizes a floating mechanism to accommodate misalignments when the medical device is mounted.

It is another advantage of the present disclosure to allow freedom of motion for a device connector to prevent damage to the device connector and/or the medical device when the medical device is mounted to the hub connectivity station.

It is a further advantage of the present disclosure to enable easier mounting of a medical device to a hub connectivity station.

It is an additional advantage of the present disclose to enable attachment and/or separation of a docking apparatus from another docking apparatus 106 without the use of tools.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a diagram of a perspective view of a hub connectivity station with a single docking apparatus and a connectivity stage, according to an example embodiment of the present disclosure.

FIG. 2 is a diagram of an assembly view of the hub connectivity station of FIG. 1, according to an example embodiment of the present disclosure.

FIG. 3 shows a bottom view of the hub connectivity station of FIG. 1, according to an example embodiment of the present disclosure.

FIG. 4 is a diagram of a hub connectivity station with six docking apparatuses, according to an example embodiment of the present disclosure.

FIG. 5 is a diagram of the hub connectivity station of FIGS. 1 to 3 with two medical devices connected to the docking apparatus, according to an example embodiment of the present disclosure.

FIG. 6 is a diagram of the hub connectivity station of FIGS. 1 to 3 illustrating an exploded view of the device connector assembly, according to an example embodiment of the present disclosure.

FIG. 7 is a diagram of the hub connectivity station of FIGS. 1 to 3 illustrating a second exploded view of the device connector assembly, according to an example embodiment of the present disclosure.

FIG. 8 shows a cross-sectional view of the hub connectivity station of FIGS. 1 to 3 illustrating details of the device connector assembly, according to an example embodiment of the present disclosure.

FIG. 9 is a diagram of the hub connectivity station of FIGS. 1 to 3 illustrating components of a docking apparatus connection feature, according to an example embodiment of the present disclosure.

FIG. 10 is a diagram of the hub connectivity station of FIGS. 1 to 3 illustrating components of a medical device latching feature, according to an example embodiment of the present disclosure.

FIG. 11 is a diagram of the hub connectivity station of FIGS. 1 to 3 illustrating additional components of the medical device latching feature, according to an example embodiment of the present disclosure.

### DETAILED DESCRIPTION

The present disclosure relates in general to a method, system, and apparatus for power management of a hub connectivity station. As disclosed herein, the hub connectivity station comprises a number of stages that are linked together in a stacked configuration. Each hub connectivity station includes a single connectivity stage that is communicatively coupled to a medical network. Additionally, each hub connectivity station includes one or more medical device docking apparatuses. Each docking apparatus can accommodate, in some embodiments, two medical devices, such as infusion pumps. The number of docking apparatuses used in the hub connectivity station depends on the number of medical devices needed for a patient treatment. Together, the connectivity stage and the docking apparatuses provide communication and power routing.

In contrast to the hub connectivity station disclosed herein, known multi-channel infusion pumps include a single controller and two to six pumps. The controller provides centralized management of the infusion pumps such that the pumps cannot be removed during operation. Additionally, the controller is configured to operate with only one type of infusion pump, thereby limiting system flexibility.

The hub connectivity station overcomes at least some of the limitations of known multi-channel infusion pumps by enabling multiple docking apparatus to be stacked, where each docking apparatus can accommodate one of many different types of medical devices. Additionally, the medical devices are configured to operate independently and may be removed from the hub connectivity station as needed, even during a treatment without interruption. Such a configuration provides a scalable, flexible, and adaptable system that concentrates medical devices into a relatively small footprint.

### Hub Connectivity Station Embodiment

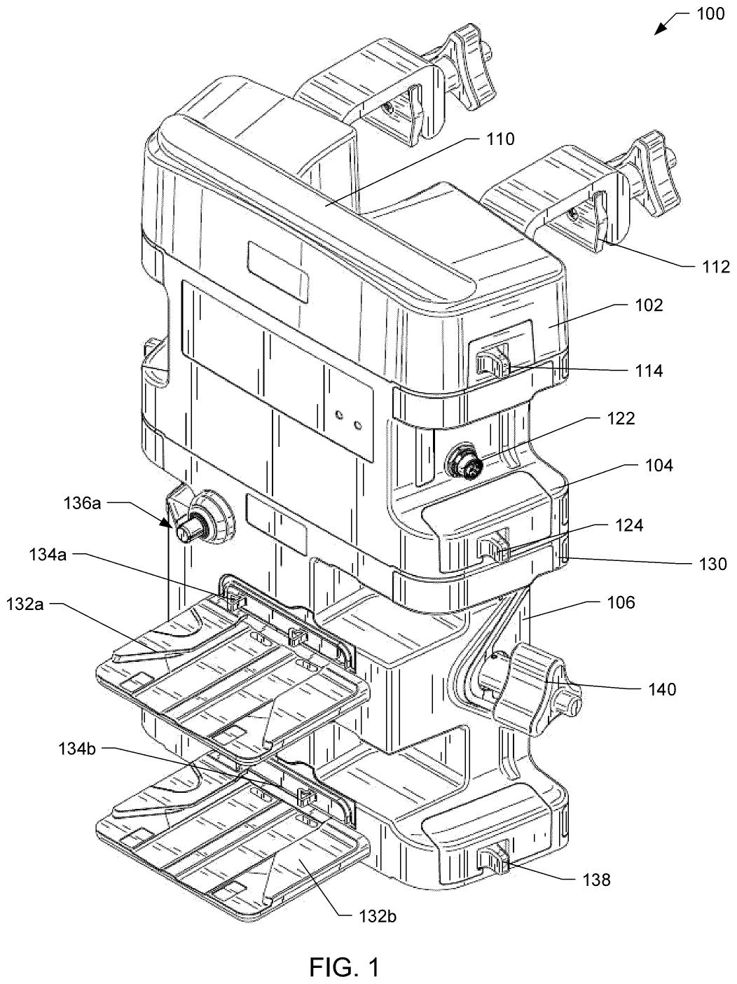

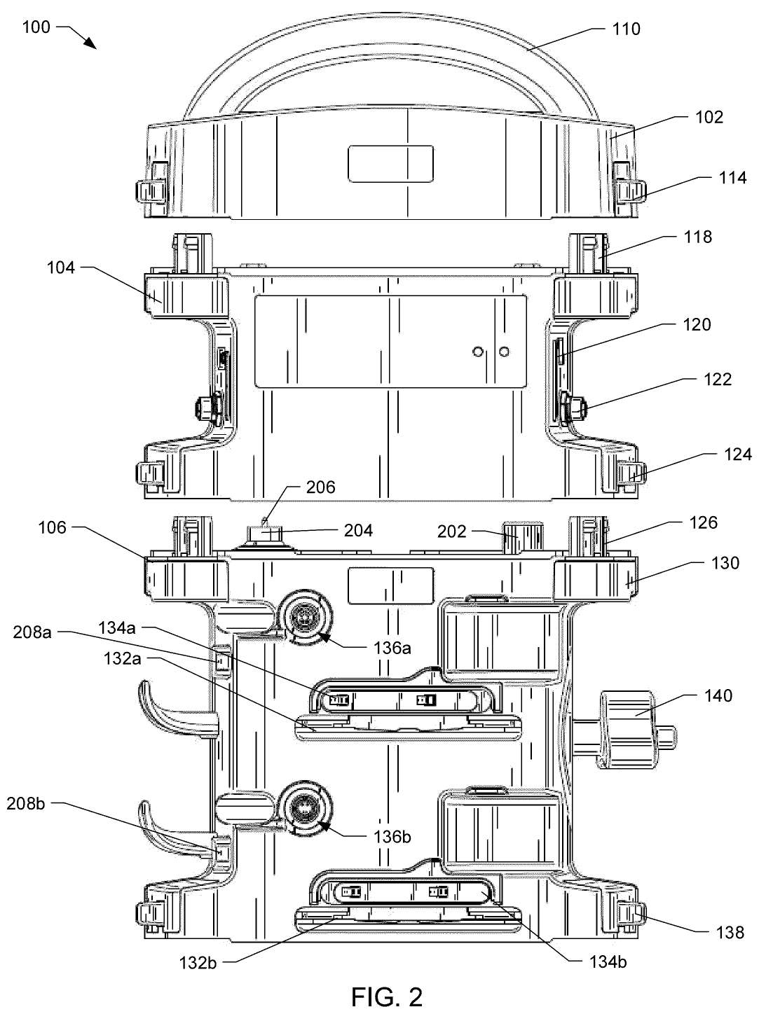

FIGS. 1 and 2 are diagrams of a hub connectivity station 100, according to an example embodiment of the present disclosure. FIG. 1 shows the hub connectivity station 100 from a perspective view. FIG. 2 shows the hub connectivity station 100 from a front assembly view.

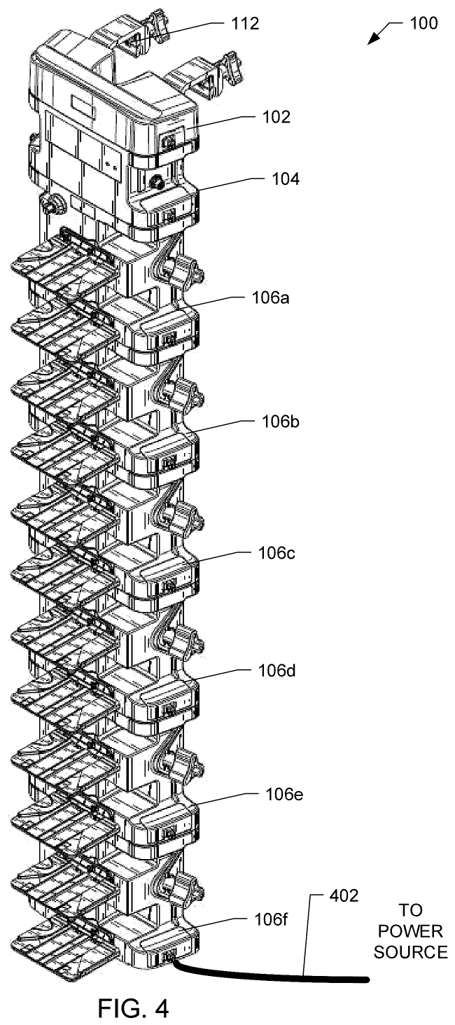

The hub connectivity station 100 includes a handle stage 102, a connectivity stage 104, and a medical device docking apparatus 106. While only one medical device docking apparatus 106 is shown in FIGS. 1 and 2, the hub connectivity station 100 can include additional medical device docking apparatuses 106, as shown in FIG. 4. In some embodiments, the handle stage 102 may be omitted.

As shown in FIGS. 1 and 2, the handle stage 102 includes a handle 110, a bedrail clamp 112, and a release lever 114. The handle 110 is configured to enable the hub connectivity station 100 to be carried by a clinician. The example handle 110 has a semi-circular shape that extends vertically from a housing of the handle stage 102. The handle 110 may be connected to or integrally formed with the housing of the handle stage 102 to support the weight of the connectivity stage 104 and the one or more docking apparatuses 106 during transportation to other locations within a hospital or other medical environment.

The bedrail clamp 112 includes a bracket and a release knob to enable the hub connectivity station 100 to be connected to railing or panel of a patient's bed. The release knob is rotated in one direction to cause a screw or other actuator to move to a closed position, thereby tightening against a rail or hospital bed panel. The release knob is rotated in an opposite direction to cause the screw or other actuator to move to a closed position. In some embodiments, the bedrail clamp 112 may be omitted.

The release lever 114 is configured, when actuated by a clinician, to enable the handle stage 102 to separate from the connectivity stage 104. As shown in FIG. 2, the connectivity stage 104 includes a protrusion section 118 that is configured to mate with a recess section within the handle stage 102. The protrusion section 118 may include a tab or other mechanical connector that engages the corresponding release lever 114 and slots or protrusions within the recess section of the handle stage 102. Insertion of the protrusion section 118 into the recess section may create a secure connection between the handle stage 102 and the connectivity stage 104 when the release lever 114 is engaged. However, when the release lever 114 is actuated, the protrusion section 118 may be slid from the recess section of the handle stage 102, thereby enabling separation of the connectivity stage 104. While FIGS. 1 and 2 show the release lever 114 located on both sides of the handle stage 102, in other embodiments the release lever 114 is located only on one side of the handle stage 102.

The connectivity stage 104 is configured to provide communication between the hub connectivity station 100 and a hospital network. A housing of the connectivity stage 104 includes an Ethernet port 120 (shown in FIG. 2), such as an RJ45 port. The connectivity stage 104 also includes one or more M12 connectors 122. In some embodiments, the M12 connector 122 is omitted. Similar to the handle stage 102, the connectivity stage 104 also includes a release lever 124 to enable the docking apparatus 106 to be removably connected via similar protrusion sections 126.

The docking apparatus 106 is configured to be connected to a bottom of the housing of the connectivity stage 104, as shown in FIGS. 1 and 2. In the illustrated example, the docking apparatus 106 is configured to connect to two medical devices, such as infusion pumps. In other embodiments, the docking apparatus 106 may include a single shelf and connector to couple to only one medical device or have as many as six shelves and six connectors to couple to six medical devices.

The docking apparatus 106 includes a housing 130 and shelves 132a, 132b that extend from the housing 130. Latches 134a, 134b are respectively positioned above each of the shelves 132a, 132b. The latches 134a, 134b are configured to releasably couple to respective medical devices, such as infusion pumps. The shelves 132a, 132b are configured to support a weight of a respective medical device to ensure the medical device does not break away from the respective latch 134a, 134b. The width of the shelves 132a, 132b is configured to correspond to a width of a largest medical device that is to be connected to the docking apparatus 106 such that the largest medical device does not have a significant portion that extends beyond the width of the shelves 132a, 132b. The shelves have a length that is between 50% to 75% of a length of the docking apparatus 106 to provide sufficient support for a connected medical device. Additionally, as shown in FIGS. 1 and 2, the shelves 132a, 132b include recessed sections to guide the placement of a medical device into the respective latch 134a, 134b.

The docking apparatus 106 also includes two device connector assemblies 136a, 136b that extend from the housing 130. The device connector assemblies 136a, 136b are located on the housing 130 of the docking apparatus 106 so as to mate with respective connectors on compatible medical devices. In the illustrated example, the device connector assemblies 136a, 136b have a cylindrical shape that protrudes from the housing 130. In other examples, the device connector assemblies 136a, 136b may be ports that are recessed into the housing 130 and/or have a rectangular or hexagonal shape. Further, while the device connector assemblies 136a, 136b are shown as being located above the respective latches 134a, 134b, in other embodiments, they may be offset from the latches 134a, 134b.

The device connector assemblies 136a, 136b are configured to include pins or other contacts for power and data routing between a medical device and the docking apparatus 106. In an example, the device connector assemblies 136a, 136b include a device-present pin for detecting the connection of a medical device. Circuitry within the medical device may apply a low voltage or ground to the device-present pin when the medical device is connected, for example, to the device connector assembly 136a. The docking apparatus 106 is configured to detect when the device-present pin receives a low-voltage or ground to determine when the device is connected.

The device connector assemblies 136a, 136b may also include pins for a controller area network (“CAN”) connection (e.g., a CAN high signal pin and a CAN low signal pin) and/or an Ethernet connection (e.g., positive and negative transmission and receiving signal pins). In some embodiments, the device connector assemblies 136a, 136b may include an identifier request pin. In these embodiments, the identifier request pin is used by the docking apparatus 106 and/or the connectivity stage 104 to transmit a signal or a message requesting that the medical device provide a CAN identifier (e.g., a CAN identification Query ID) through the CAN connection.

FIGS. 1 and 2 also show the housing 130 of the docking apparatus 106 includes a release lever 138, which is similar to the release lever 124 of the connectivity stage 104. Similar to the respective connections between the handle stage 102 and the connectivity stage 104 and the connectivity stage 104 and the docking apparatus 106, the docking apparatuses 106 may be removably connected to each other in a stacked arrangement. The release lever 138 enables the docking apparatus 106 to be disconnected from a lower-positioned docking apparatus.

As discussed above, the hub connectivity station 100 is configured to route power and data through the stack of the connectivity stage 104 and one or more docking apparatuses 106. FIG. 2 shows connectors that are positioned at a top of the housing 130 of the docking apparatus 106. The connectors include an AC outlet connector 202 and a top data connector 204. The AC outlet connector 202 is configured to protrude from a top of the housing 130 and may include an International Electrotechnical Commission (“IEC”) connector with countersunk holes. The connector 202 may have 10 ampere, 250 AC voltage rating.

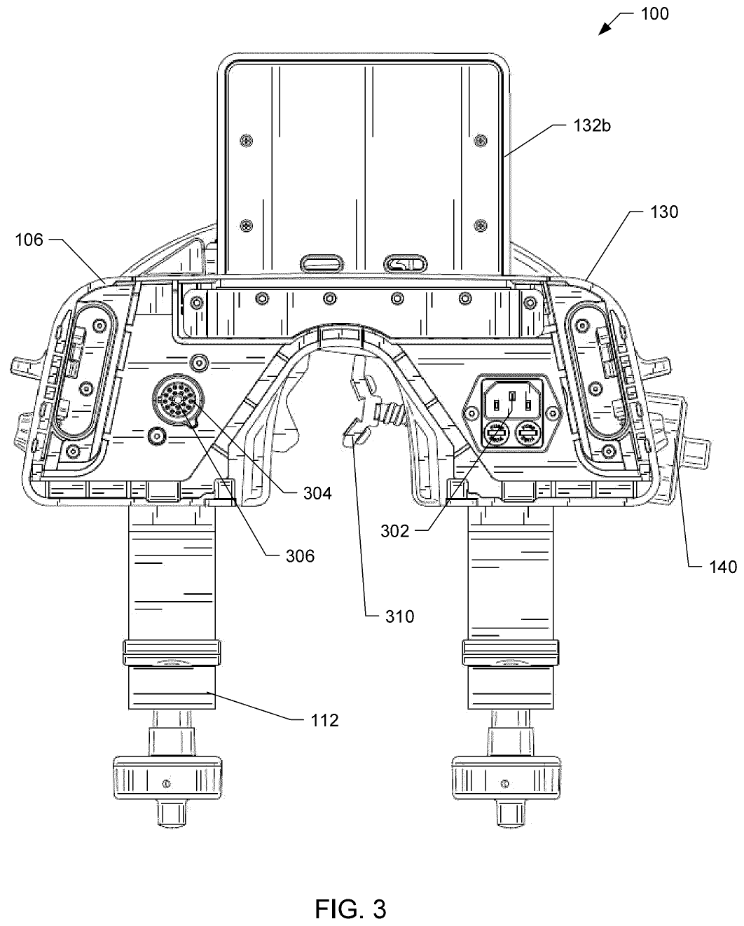

The AC outlet connector 202 is configured to connect to a corresponding AC inlet connector that is provided at a bottom of the connectivity stage 104 and other docking apparatuses 106. FIG. 3 shows a bottom view of the docking apparatus 106, according to an example embodiment of the present disclosure. It should be appreciated that the bottom view is similar for the connectivity stage 104. As shown, a bottom of the housing 130 of the docking apparatus 106 includes an AC inlet connector 302 that is aligned with the AC outlet connector 202 at the top of the housing. The AC inlet connector 302 may include a panel mounted appliance inlet that sits within a recess section of the docking apparatus 106. When connected, the prongs of the AC inlet connector 302 slide into the apertures of the AC outlet connector 202 as a body of the AC outlet connector 202 fits within a recess of the AC inlet connector 302.

The aligned positioning of the AC inlet connector 302 and the AC outlet connector 202 enable the connectivity stage 104 and the docking apparatuses 106 to be electrically connected together when stacked within the hub connectivity station 100. Further, the consistent positioning of the AC inlet connector 302 and the AC outlet connector 202 enable any docking apparatus 106 to be connected to any other docking apparatus 106 or the connectivity stage 104. In some alternative embodiments, the AC inlet connector 302 may be positioned on the top of the housing 130 while the AC outlet connector 202 is positioned on the bottom of the housing 130 of the docking apparatus 106. Further, while the connectors 202 and 302 are shown as Type-B connectors for use in North America, in other examples the connectors may be of Type C through Type 0 to enable the hub connectivity station 100 to be used in other parts of the world.

To power the hub connectivity station 100, a power cord is connected to the AC inlet connector 302 at the bottom of a lower-most docking apparatus 106. An opposite end of the power cord may be connected to an electrical outlet, a power rail, a battery, a generator, or any other power source. Such a configuration ensures that the power cord is placed as close to the ground as possible to reduce the number of wires and lines at higher sections of the station 100. Further, the routing of power through the hub connectivity station 100 means that only a single power cord is needed.

In addition to routing power throughout, the hub connectivity station 100 is also configured to enable data to be communicated among the connectivity stage 104 and the docking apparatuses 106. FIG. 2 shows the top data connector 204 adjacent to the AC outlet connector 202 at the top of the housing 130 of the docking apparatus 106. FIG. 3 shows a bottom data connector 304 that is located at the bottom of the housing 130 adjacent to the AC inlet connector 302. The top data connector 204 is configured to connect to a bottom data connector 304 of the connectivity stage 104 or the bottom data connector 304 of another docking apparatus 106.

The data connectors 204, 304 may include pins for CAN communication. In some embodiments, the data connectors 204, 304 may additionally include pins for universal asynchronous receiver-transmitter (“UART”) communication and/or Ethernet communication between the connectivity stage 104 and the stacked docking apparatuses 106 within the hub connectivity station 100. The data connectors 204, 304 each include at least one respective pin 206, 306 to detect when the connectivity stage 104 or another docking apparatus 106 is connected. In some embodiments, the pin 306 of the bottom data connector 304 is electrically coupled to ground or a low voltage. As described in more detail below, a circuit with the docking apparatus 106 is configured to detect when the pin 206 of the top data connector 204 is pulled to the ground or low voltage to detect when another docking apparatus 106 or the connectivity stage 104 is connected.

FIG. 2 also shows that the docking apparatus 106 includes medical device release buttons 208a, 208b. The medical device release buttons 208a, 208b are configured to decouple the respective medical devices from the latches 134a, 134b. FIGS. 1 to 3 also show that the docking apparatus 106 includes a pole clamp, which includes a knob 140 and an actuator 310. The pole clamp enables the docking apparatus 106 to securely couple the hub connectivity station 100 to a pole. Since each docking apparatus 106 includes a pole clamp, multiple stacked docking apparatuses 106 are connected to the same pole to securely connect the hub connectivity station 100 when a plurality of medical devices are used. To connect, the knob 140 is rotated, which causes the actuator 310 to engage the pole. To release, the knob 140 is rotated in an opposite direction, which causes the actuator 310 to move away from the pole.

FIG. 4 shows a diagram of the hub connectivity station 100 of FIG. 1 with six docking apparatuses 106a to 106f stacked below the connectivity stage 104, according to an example embodiment of the present disclosure. In the illustrated example, the handle stage 102 is provided at the top of the hub connectivity station 100. The connectivity stage 104 is connected to the bottom of the handle stage 102. A first docking apparatus 106a is connected to a bottom of the connectivity stage 104. A second docking apparatus 106b is connected to a bottom of the first docking apparatus 106a. Further, the third through sixth docking apparatuses 106c to 106f are sequentially stacked. A power cord 402 electrically couples the sixth docking apparatus 106f to a power source, such as a wall outlet.

The illustrated six docking apparatuses 106a to 106f are configured to accommodate twelve medical devices. To support the weight of the docking apparatuses 106a to 106f themselves in addition to the medical devices, each of the docking apparatuses 106a to 106f include pole clamps for connecting to a pole. In the alternative, the bedrail clamp 112 may connect to a bedrail to support the hub connectivity station 100.

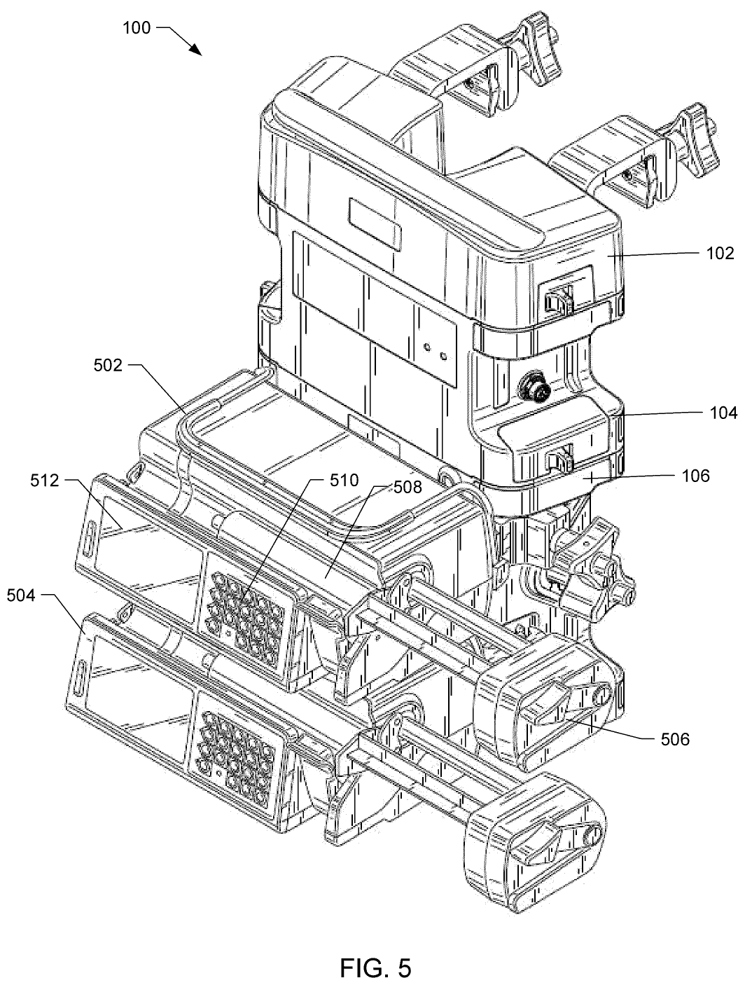

FIG. 5 is a diagram of the hub connectivity station 100 of FIGS. 1 to 3 with two medical devices 502, 504 connected to the docking apparatus 106, according to an example embodiment of the present disclosure. As shown, a first medical device 502 is slide across the shelf 132a to engage the latch 134a. As the medical device 502 is being connected, the device connector assembly 136a of the docking apparatus 106 contacts a corresponding connector of the medical device 502. In a similar manner, a second medical device 504 is slid across the shelf 132b to engage the latch 134b. As the medical device 504 is being connected, the device connector assembly 136b of the docking apparatus 106 contacts a corresponding connector of the medical device 504.

In the illustrated example, the medical devices 502 and 504 are syringe pumps, which include an actuator 506 that presses on a plunger of a syringe 508 to dispense a fluid into an IV tube. The actuator 506 is controlled by a motor within a housing of the medical device 502. Additionally, the medical device 502 includes a user interface comprising a keypad 510 and a display screen 512. The keypad 510 includes one or more buttons switches for controlling operation of the medical device 502. The display screen 512 displays graphics and text regarding the operation of the medical device 502. In some embodiments, the display screen 512 may be a touchscreen. In some embodiments, a clinician uses the keypad 510 and the display screen 512 program an infusion therapy. In addition to manual programming, the medical device 502 may receive electronic prescriptions from a hospital information system via a network. The medical device 502 may include one or more drug libraries that include particular limits based on care area, dose, rate of change, drug type, concentration, patient age, patient weight, etc.

As an infusion pump, the medical device 502 is configured to perform an infusion therapy for a patient, which includes the infusion of one or more fluids, solutions, or drugs into the patient. The medical device 502 operates according to an infusion prescription entered by a clinician at the keypad 510 and/or display screen 512 or received via a network. The medical device 502 may compare the prescription to the drug library and provide any alerts or alarms when a parameter of the prescription violates a soft or hard limit. The medical device 502 is configured to monitor the progress of the therapy and periodically transmit infusion therapy progress data to a gateway server. The infusion therapy progress data may include, for example, an infusion rate, a dose, a total volume infused, a time remaining for the therapy, a fluid concentration, a rate change, a volume remaining within a medication container, a fluid name, a patient identifier, titration information, bolus information, a care area identifier, a timestamp when the data was generated, an alarm condition, an alert condition, an event, etc. In some instances, the infusion therapy progress data includes a new infusion start event including information indicative of an infusion pump identifier, an infused fluid name, an infusion rate, a volume to be infused, a dose, a volume remaining, and/or a time the new infusion start event was generated by the infusion pump. The medical device 502 may transmit the data continuously, periodically (e.g., every 30 seconds, 1 minute, etc.), or upon request by the gateway server. While a syringe pump is shown, the docking apparatus 106 may also connect to other types of infusion pumps such as linear peristaltic pumps, large volume parenteral pumps, ambulatory pumps, PCA pumps, multi-channel pumps, etc.

The medical devices 502 and 504 may also include renal failure therapy (“RFT”) machines, which may include any hemodialysis, hemofiltration, hemodiafiltration, continuous renal replacement therapy (“CRRT”), or peritoneal dialysis machine. CRRT is a dialysis modality typically used to treat critically ill, hospitalized patients in an intensive care unit who develop acute kidney injury (“AKI”). Unlike chronic kidney disease, which occurs slowly over time, AKI often occurs in hospitalized patients and typically occurs over a few hours to a few days. A patient, undergoing hemodialysis, for example, is connected to the RFT machine, where the patient's blood is pumped through the machine. The blood passes through a dialyzer of the machine, which removes waste, toxins and excess water (e.g., ultrafiltrate) from the blood. The cleaned blood is returned to the patient.

Hemodialysis is a renal failure treatment in which waste from the blood is diffused across a semi-permeable membrane. During hemodialysis, blood is removed from the patient and flows through a semi-permeable membrane assembly (dialyzer), where the blood flows generally counter-current to dialysis solution flowing on the other side of the semipermeable membrane. In the dialyzer, toxins from the blood travel across the semi-permeable membrane and exit the dialyzer into used dialysis solution (dialysate). The cleaned blood having flowed through the dialyzer is then returned to the patient.

Hemofiltration is another renal failure treatment, similar to hemodialysis. During hemofiltration, a patient's blood is also passed through a semipermeable membrane (a hemofilter), where fluid (including waste products) is pulled across the semipermeable membrane by a pressure differential. This convective flow brings certain sizes of molecular toxins and electrolytes (which are difficult for hemodialysis to clean) across the semipermeable membrane. During hemofiltration, a replacement fluid is added to the blood to replace fluid volume and electrolytes removed from the blood through the hemofilter. Hemofiltration in which replacement fluid is added to the blood prior to the hemofilter is known as pre-dilution hemofiltration. Hemofiltration in which replacement fluid is added to the blood after the hemofilter is known as post-dilution hemofiltration.

The RFT machine can alternatively be a hemodiafiltration machine. Hemodiafiltration is a further renal failure treatment that uses hemodialysis in combination with hemofiltration. Blood is again pumped through a dialyzer, which accepts fresh dialysis fluid unlike a hemofilter. With hemodiafiltration, however, replacement fluid is delivered to the blood circuit, like with hemofiltration. Hemodiafiltration is accordingly a neighbor of hemodialysis and hemofiltration.

Alternatively, the medical device 502 may be a peritoneal dialysis machine, which may perform various types of peritoneal dialysis therapies, including continuous cycling peritoneal dialysis (“CCPD”), tidal flow automated peritoneal dialysis (“APD”), and continuous flow peritoneal dialysis (“CFPD”). Peritoneal dialysis infuses dialysate into a patient during fill cycles.

For any dialysis treatment, the RFT machine may compare parameters of a prescription to one or more limits and provide any alerts or alarms when a parameter of the prescription violates a soft or hard limit. The RFT machine is configured to monitor the progress of the therapy and periodically transmit dialysis therapy progress data to a gateway server. The dialysis therapy progress data may include, for example, a fill rate, a dwell time, a drain or fluid removal rate, a blood flow rate, an effluent dose, an ultrafiltration removal rate, a dialysate removal rate, a total dialysate infused, a dialysate flow, a replacement pre-flow, a replacement post-flow, a patient weight balance, a return pressure, an excess patient fluid sign, a filtration fraction, a time remaining, a dialysate concentration, a dialysate name, a patient identifier, a room identifier, a care area identifier, a timestamp when the data was generated, an alarm condition, an alert condition, an event, etc. The RFT machine may transmit the data continuously, periodically (e.g., every 30 seconds, 1 minutes, etc.), or upon request by the gateway server.

In some embodiments, the medical device 502 may include a physiological sensor. For example, the medical device 502 may include a pulse oximetry sensor. Additionally or alternatively, the physiological sensor may include a blood pressure sensor, a patient weight scale, a glucose sensor, a cardiac monitor, etc. In some instances, instead of connecting directly to the docking apparatus 106, the physiological sensor connects to the medical device 502.

In further embodiments, the docking apparatus 106 may also connect to a hemodynamic monitor, which is configured to display information relevant to hemodynamic monitoring and management. This includes fluid balance information, hemodynamic assessment information, hemodynamic parameters, and/or alerts related to infiltration, infusion line occlusions, and/or a fluid bag being near-empty or empty. The hemodynamic monitor uses therapy progress data and/or dialysis therapy progress data in addition to physiological sensor data to determine and/or display hemodynamic information.

### Hub Connectivity Station Device Connector Embodiment

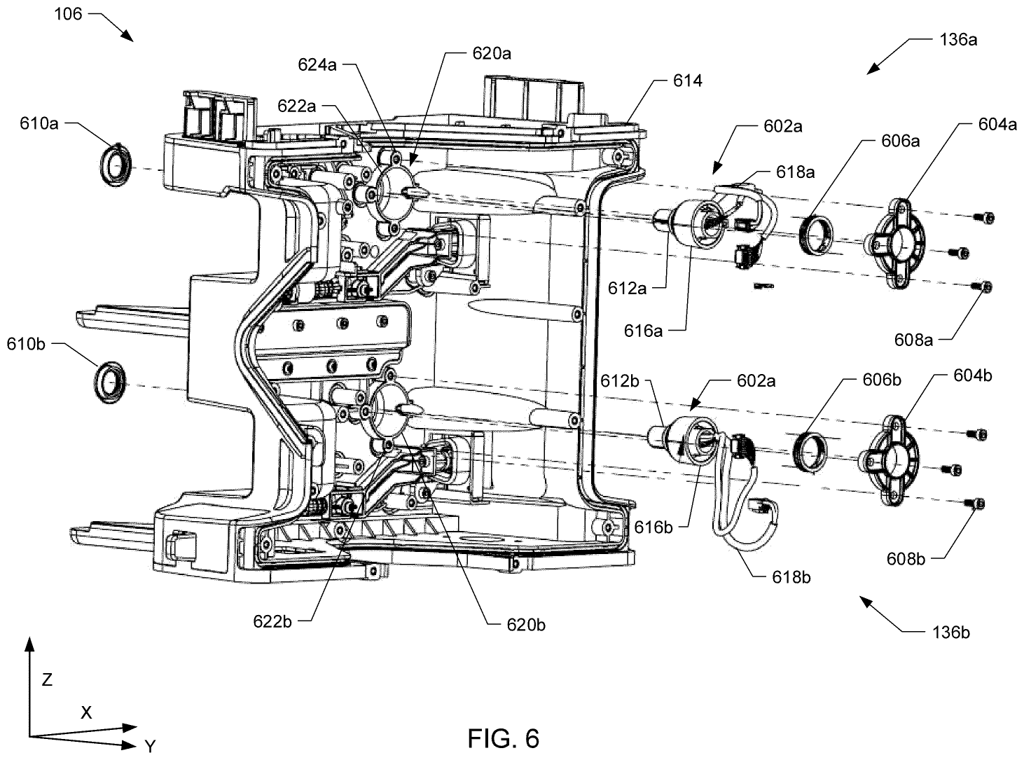

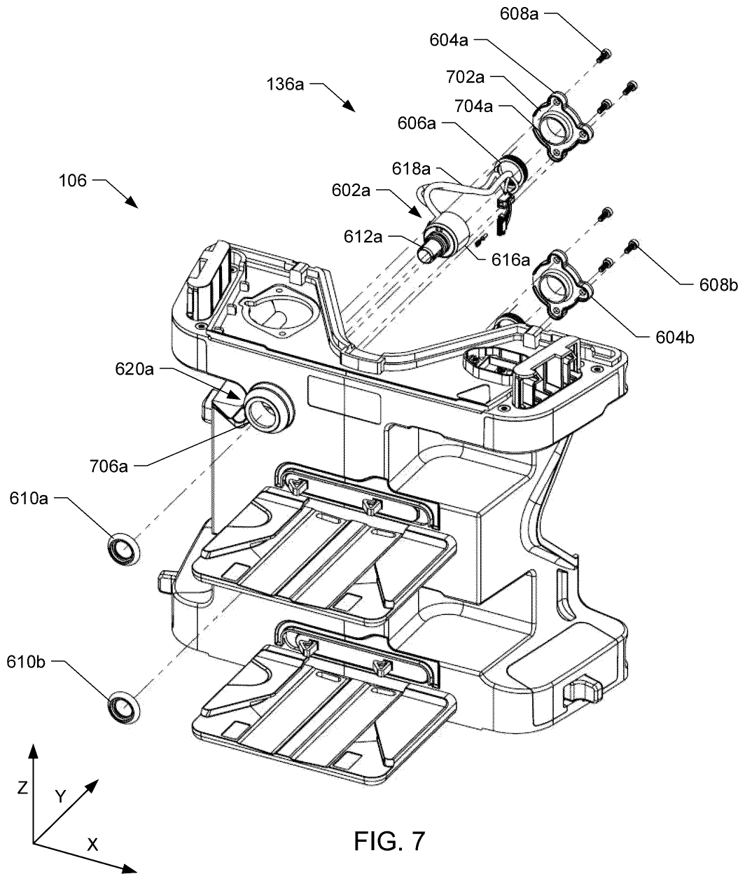

FIGS. 6 and 7 are diagrams illustrating details of the device connector assemblies 136a, 136b of the docking apparatus 106, according to an example embodiment of the present disclosure. FIG. 6 shows a first exploded view of the device connector assemblies 136a, 136b from a rear perspective of the docking apparatus 106. FIG. 7 shows a second exploded view of the device connector assemblies 136a, 136b, from a rear view.

The example device connector assembly 136a includes device connector 602a, mounting bracket 604a, spring 606a, mounting hardware 608a, and a seal 610a. The example device connector 602a is configured to receive a connector of a medical device which is mounted to the docking apparatus 106. The example device connector 602 thus provides power and/or data connectivity between a medical device and the docking apparatus 106.

The example device connector 602a includes a cylindrical portion 612a. When the device connector assembly 136a is installed in the docking apparatus 106, at least a portion of the cylindrical portion 612a of the device connector 602a protrudes outward from an exterior surface of a housing 614 of the docking apparatus 106. The example device connector 602a includes a base portion 616a. The example base portion 616a is concentric with the cylindrical portion 612a and has a diameter greater than a diameter of the cylindrical portion 612a. When the device connector assembly 136a is installed in the docking apparatus 106, the base portion 616a remains interior of an inside surface of the housing 614 of the docking apparatus 106.

The example device connector 602a further includes connector wires 618a. The example connector wires 618a provide power and/or data routing between a medical device connected to the device connector assembly 136a and the docking apparatus 106. Further, the example connector wires 618a may transmit data corresponding to a device-present pin included in the device connector 602a as described above. When the device connector assembly 136a is installed in the docking apparatus 106, the connector wires 618a remain interior of an inside surface of the housing 614.

The example device connector assembly 136a further includes the mounting bracket 604a. The example mounting bracket 604a is configured to be fixedly coupled to an interior surface of the housing 614 of the docking apparatus 106. The example mounting bracket 604a includes a flange portion 702a and a retainer portion 704a protruding from the flange portion 702a.

The example mounting bracket 604a may be coupled to the housing 614 using the mounting hardware 608a. The example mounting hardware may include one or more of bolts, screws, rivets, or any other hardware suitable for fixedly coupling the mounting bracket 604a to the housing 614. The example mounting bracket 604a includes one or more openings in the flange portion 702a to receive the mounting hardware 608a. When the example device connector assembly 136a is installed in the docking apparatus 106, the mounting bracket 604a is located interior (e.g., rearward, at a greater position on the y-axis) of the device connector 602a. As such, the example mounting bracket 604a restricts movements of the device connector 602a in the y-direction.

The example device connector assembly 136a further includes the spring 606a. The example spring 606a includes a circular compression spring such as a wave spring, a single turn wave spring, a multi-turn wave spring, or a conventional compression spring. When the example device connector assembly 136a is installed in the docking apparatus 106, the example spring 606a is located between the flange portion 702a of the mounting bracket 604a and a rear surface of the device connector 602a. Additionally, the example spring 606a is located around the retainer portion 704a of the example mounting bracket 604a. The example retainer portion 704a restricts movement of the spring 606a in x- and z-directions while the flange portion 702a of the mounting bracket 604a limits movement of the spring 606a in the y-direction.

The example spring 606a is configured to apply force to the rear surface of the device connector 602a. For example, when the device connector assembly 136a is installed in the docking apparatus 106, a space between the exterior surface of the mounting bracket 604a and the rear surface of the device connector 602a may be less than an uncompressed height of the spring 606a. As such, when the device connector assembly 136a is installed, the spring 606a is compressed in order to fit in the space. Accordingly, because the spring 606a is limited in movement in the y-direction by the mounting bracket 604a, the spring 606a applies a force to the rear surface of the device connector 602a. The force applied to the rear surface of the device connector 602a urges the device connector 602a toward the interior surface of the housing 614 of the docking apparatus 106.

The example device connector assembly 136a further includes the seal 610a. When the device connector assembly 136a is installed in the docking apparatus, the example seal 610a is located around the portion of the cylindrical portion 612a of the device connector 602a which protrudes from the exterior surface of the housing 614. The example seal 610a may be coupled to the cylindrical portion 612a of the device connector 602a and located proximate the exterior surface of the housing 614. As such, the example seal 610a may restrict ingress (e.g., movement in a positive y-direction) of the device connector 602 into the housing 614. Further, the example seal 610a may prevent debris from entering the inside of the housing 614.

The housing 614 of the example docking apparatus 106 includes a connector aperture 620a. The example connector aperture 620a comprises a tapered opening in the housing 614. The tapered opening of the connector aperture 620a is configured to receive the device connector 602a. For example, an exterior portion 706a of the connector aperture 620a is sized to receive the cylindrical portion 612a of the device connector 602a. An interior portion 622a of the connector aperture 620a is sized to receive the larger diameter of the base portion 616a of the device connector 602a. In this manner, the example device connector 602a may be inserted through the connector aperture 620a from the interior of the docking apparatus 106 such that at least a portion of the cylindrical portion 612a protrudes outward from the exterior surface of the housing 614. Accordingly, the base portion 616a of the device connector 602a is retained on the interior of the docking apparatus 106 when the device connector assembly 136a is assembled on the housing 614.

The example housing 614 of the docking apparatus 106 includes one or more mounting holes 624a. In the example of FIG. 6, the mounting holes 624a are proximate the interior portion 622a of the connector aperture 620a. The example mounting holes 624a are configured to receive the mounting hardware 608a in order to couple the mounting bracket 604a to the housing 614.

The example docking apparatus 106 further includes device connector assembly 136b having device connector 602b, mounting bracket 604b, spring 606b, mounting hardware 608b, and a seal 610b which may be substantially the same as the respective components of device connector assembly 136a. Accordingly, the housing 614 includes connector aperture 620b and mounting holes 624b. While the example of FIG. 6 illustrates a docking apparatus 106 with two device connector assemblies 136a, 136b, an example docking apparatus may include only one device connector assembly or more than two (e.g., three, four, etc.) device connector assemblies.

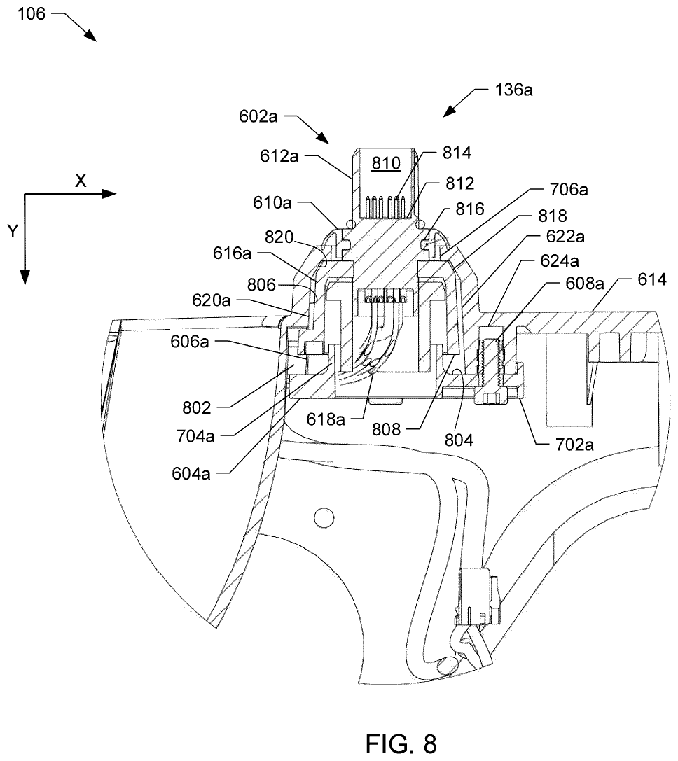

FIG. 8 shows a cross-sectional view of a portion the docking apparatus 106 of FIGS. 1 to 3 illustrating details of the device connector assembly 136a, according to an example embodiment of the present disclosure. FIG. 8 illustrates the device connector assembly 136a installed in the housing 614 of the docking apparatus. In the example of FIG. 8, no medical device is mounted (e.g., connected) to the device connector assembly 136a.

In FIG. 8, the example mounting bracket 604a is fixedly coupled to the housing 614 using the mounting hardware 608a. For example, the mounting hardware 608a passes through an opening in the flange portion 702a of the mounting bracket 604a and into one of the mounting holes 624a. In the example of FIG. 8, the mounting hardware 608a comprises a threaded screw and the mounting hole 624a is internally threaded to receive the screw. While a single mounting hardware 608a is shown in the view of FIG. 8, it should be appreciated that the mounting bracket 604a may be coupled to the housing 614 using two or more of the mounting hardware 608a.

When the example mounting bracket 604a is coupled to the housing 614, a mounting cavity 802 is formed between a flange surface 804 of the mounting bracket 604a and an interior surface 806 of the connector aperture 620a. The example device connector 602a is positioned in the mounting cavity 802. Accordingly, the example device connector 602a is not fixedly connected to the housing 614, but only restricted in movement by the size and shape of the mounting cavity 802.

The example spring 606a is also positioned within the mounting cavity 802. The example spring 606a is positioned around the retainer portion 704a and between the flange surface 804 of the mounting bracket 604a and a rear surface 808 of the device connector 602a. When the example device connector assembly 136a is assembled in the housing 614, the example spring 606a is compressed such that the spring 606a applies a force on the rear surface 808 of the device connector 602a. The force urges the device connector 602a toward the interior surface 806 of the connector aperture 620a.

In the example of FIG. 8, the cylindrical portion 612a and the base portion 616a of the device connector 602a are formed separately and fixedly connected. In some examples, the device connector 602a comprises a unitary body which includes both the cylindrical portion 612a and the base portion 616a.

As illustrated in FIG. 8, the cylindrical portion 612a of the device connector 602a includes a connector cavity 810. The example connector cavity 810 is configured to receive at least a portion of a connector of a medical device which is to be mounted to the docking apparatus 106. Protruding from a base 812 of the connector cavity 810 are one or more pins 814. The example one or more pins 814 may include a device-present pin, pins for a CAN connection, an Ethernet connection, and/or an identifier request pin, as described above. The example one or more pins 814 are configured to provide electrical connectivity between a connector of a medical device and the device connector 602a. Data and/or power may be routed between the docking apparatus 106 and a medical device through the wires 618a and the one or more pins 814.

The example device connector 602a includes a circumferential groove 816. The circumferential groove is configured to receive a portion of the seal 610a in order to couple the seal 610a to the device connector 602a. The example seal 610a may be installed in the groove 816 of the device connector 602a after installation of the device connector 602a into the housing 614. In this manner, the example seal 610a may prevent entrance of debris into the docking apparatus 106 via the exterior portion 706a of the connector aperture 620a.

The example base portion 616a of the device connector 602a includes a connector tapered surface 818. The example connector tapered surface 818 is positioned at a front portion of the base portion 616a and includes an angled surface which reduces the diameter of the base portion 616a. Correspondingly, the example connector aperture 620a includes an aperture tapered surface 820. The example aperture tapered surface 820 comprises a portion of an interior surface of the interior portion 622a of the connector aperture 620a. When the device connector assembly 136a is assembled in the housing 614, the connector tapered surface 818 is proximate to the aperture tapered surface 820.

Before a medical device is connected to the device connector assembly 136a, the device connector 602a is located at an initial position relative to the housing 614. During connection of a medical device to the device connector assembly 136a, a connector of the medical device approaches the cylindrical portion 612a of the device connector 602. For example, a medical device (e.g., the medical device 502 of FIG. 5) is slid across a shelf (e.g., the shelf 132a of FIG. 1) until the medical device engages a latch (e.g., the latch 134a of FIG. 1). As the medical device is connected, a connector of the medical device approaches and contacts the device connector 602. During this connection, at least a portion of the connector of the medical device is inserted into the connector cavity 810 and the one or more pins 814 form an electrical connection with the connector of the medical device.

In some examples, when the medical device engages a latch of the docking apparatus 106, a distal portion of the connector of the medical device extends past the initial position of the base 812 of the connector cavity 810. In these examples, the spring 606a may deform (e.g., compress) to allow the device connector 602a to translate rearward (e.g., in a positive y-direction) to accommodate the connector of the medical device. Thus, while the medical device is connected to the docking apparatus 106, the device connector 602a is located in a position other than the initial position.

In some examples, the connector of the medical device is misaligned with the device connector 602a when the medical device is being mounted to the docking apparatus. For example, a central axis of the connector of the medical device may be offset from a central axis of the device connector 602a when the medical device is slid across the shelf and/or engaged with the latch of the docking apparatus 106. In these examples, the connector of the medical device may contact an exterior portion of the cylindrical portion 612a of the device connector 602a instead of being inserted into the connector cavity 810. After such contact, as the medical device is slid closer to the docking apparatus 106, the connector of the medical device may apply a force on the device connector 602a which causes the spring 606a to compress and the device connector 602a to translate rearward.

Once the device connector 602a translates rearward (e.g., in the positive y-direction) from its initial position, the device connector 602a is further free to move in both x-and z-directions. As such, the device connector 602a has freedom to move in x-, y-, and z-directions within the mounting cavity 802. For example, the device connector 602a may translate in x-, y-, or z-directions or may rotate around the x-, y-, or z-axes.

Accordingly, the device connector 602a may move (e.g., translate and/or rotate in one or more directions) in response to mounting a medical device with a misaligned connector. Such movement may allow the device connector 602a to move such that the central axis of the device connector 602a is aligned with the central axis of the connector of the medical device. Upon such alignment, the connector of the medical device may connect to the device connector 602a. For example, the device connector 602a moves such that at least a portion of the connector of the medical device may insert into the connector cavity 810 and the one or more pins 814 form an electrical connection with the connector of the medical device.

After the medical device is removed from the docking apparatus 106, interaction of the connector tapered surface 818 and the aperture tapered surface 820 guides the device connector 602a back to the initial position. For example, after the connector of the medical device is decoupled from the device connector 602a, any compressive force (e.g., force in the positive y-direction) the connector of the medical device applied to the device connector 602a is released. In turn, force applied by the spring 606a to the rear surface 808 of the device connector 602a urges the device connector 602a in the negative y-direction. As the device connector 602a moves in the negative y-direction, the connector tapered surface 818 is guided by the aperture tapered surface 820 so that the device connector 602a returns to the initial position.

In some examples, while the design condition of the device connector assembly 136a and the housing 614 ensures mechanical and electrical connection between the medical device and the docking apparatus 106, actual part tolerances may cause an insecure mechanical and/or electrical connection. For example, there may be a gap in the y-direction between the base 812 of the connector cavity 810 and the distal portion of the connector of the medical device when the medical device is engaged with the latch of the docking apparatus 106. If this gap is too large, the mechanical connection between the connector may be insecure and/or the one or more pins 814 may not engage with the connector of the medical apparatus.

In another example, the distal portion of the connector of the medical device may extend past the initial position of the base 812 of the connector cavity 810 in an amount greater than a possible y-direction movement of the device connector. In this case, the medical device may be unable to engage with the latch 134a of the docking apparatus 106 due to obstruction of the device connector 602a. To avoid such issues, the housing 614 and the device connector assembly 136a are designed to ensure connection between the medical device and the docking apparatus 106 when accounting for part tolerances by, for example, modifying the stroke of the spring 606a and/or including one or more compressible gaskets (not pictured).

### Hub Connectivity Station Docking Apparatus Connector Embodiment

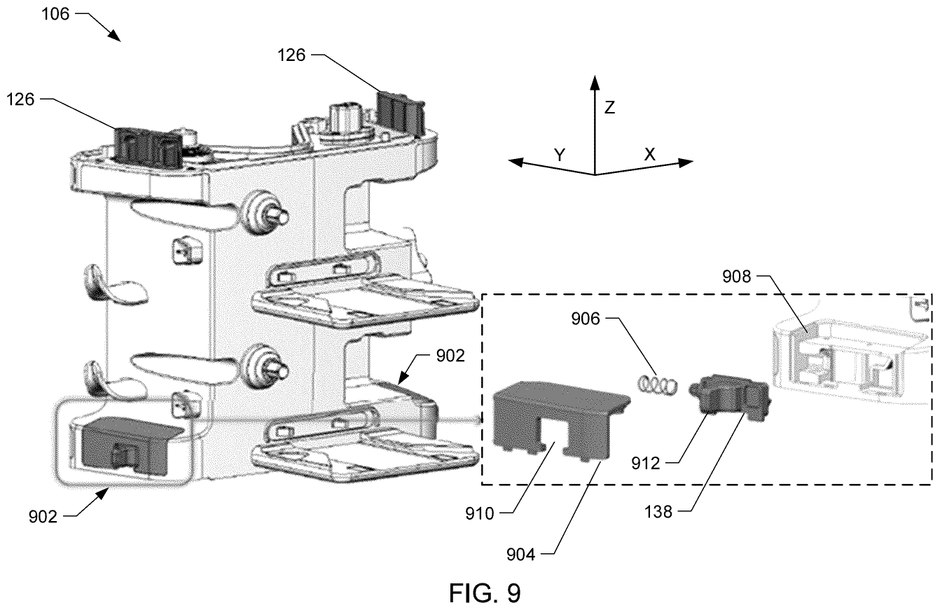

FIG. 9 is a diagram of the docking apparatus 106 of FIGS. 1 to 3 illustrating docking apparatus connection features, according to an example embodiment of the present disclosure. FIG. 9 shows the docking apparatus 106 with protrusion sections 126 and docking apparatus release mechanism 902. FIG. 9 also shows an exploded view of the docking apparatus release mechanism 902. The spring-based release mechanism illustrated in FIG. 9 allows for attachment and/or separation of a docking apparatus 106 from another docking apparatus 106 without the use of tools. For example, the release levers 138 may be actuated by the hands of a user.

As illustrated in FIG. 4, two or more docking apparatus 106 may be stacked below the connectivity stage 104 of FIG. 1. As shown in FIG. 2, the protrusion sections 126 are configured to enable a docking apparatus 106 to be removably connected to the connectivity stage 104. The protrusion sections 126 may be further configured to enable a docking apparatus 106 to be removably connected to another docking apparatus 106 in the stacked arrangement. As such, the protrusion sections 126 are configured to mate with a recess section within another docking apparatus 106. For example, the protrusion sections 126 may include a tab or other mechanical connector that engages a corresponding docking apparatus release mechanism 902 of the other docking apparatus 106 and slots or protrusions within the recess section of the other docking apparatus 106. Insertion of the protrusion sections 126 into the recess section may create a secure connection between the two docking apparatus 106 when the release lever 138 of the docking apparatus release mechanism 902 is engaged. However, when the release lever 138 of the docking apparatus release mechanism 902 is actuated, the protrusion section 126 may be slid from the recess section of the other docking apparatus 106, thereby enabling separation of the docking apparatus 106.

In the example of FIG. 9, two docking apparatus release mechanisms 902 and two protrusion sections 126 are illustrated. In this example, both of the release levers 138 of the two docking apparatus release mechanisms 902 must be actuated in order to separate the docking apparatuses 106 from one another. This configuration of docking apparatus release mechanisms 902 may reduce the occurrence of incidental release of a docking apparatus 106 from another docking apparatus 106. In other embodiments, the docking apparatus release mechanism 902 may be located on only one side of the docking apparatus 106. In further examples, two docking apparatus release mechanisms 902 may be located on the docking apparatus 106, but only one of the two must be actuated in order to separate the docking apparatuses 106 from one another.

The example docking apparatus release mechanism 902 includes a mechanism cover 904, the release lever 138, and a release spring 906. When assembled, the example release lever 138 is mounted in a release mechanism cavity 908 of the docking apparatus 106. The example release mechanism cavity 908 restricts movement of the release lever 138 in the x-and z-directions but allows movement of the release lever 138 in the y-direction. The example release spring 906 is positioned proximate to a rear portion of the release lever 138 and urges the release lever 138 in the negative y-direction. The release lever 138 may be engaged by applying a force to the release lever 138 in the positive y-direction which overcomes the force of the release spring 906, thus moving the release lever 138 in the positive y-direction.

The example mechanism cover 904 of the docking apparatus release mechanism 902 is configured to retain the release lever 138 and the release spring 906 within the release mechanism cavity 908. The example mechanism cover 904 includes an opening 910 which allows a handle portion 912 of the release lever 138 to be exposed. The opening 910 is configured to enable the release lever 138 to move in the y-direction but restrict movement of the release lever 138 in the x- and z-directions.

### Hub Connectivity Station Medical Device Latch and Release Mechanism Embodiment

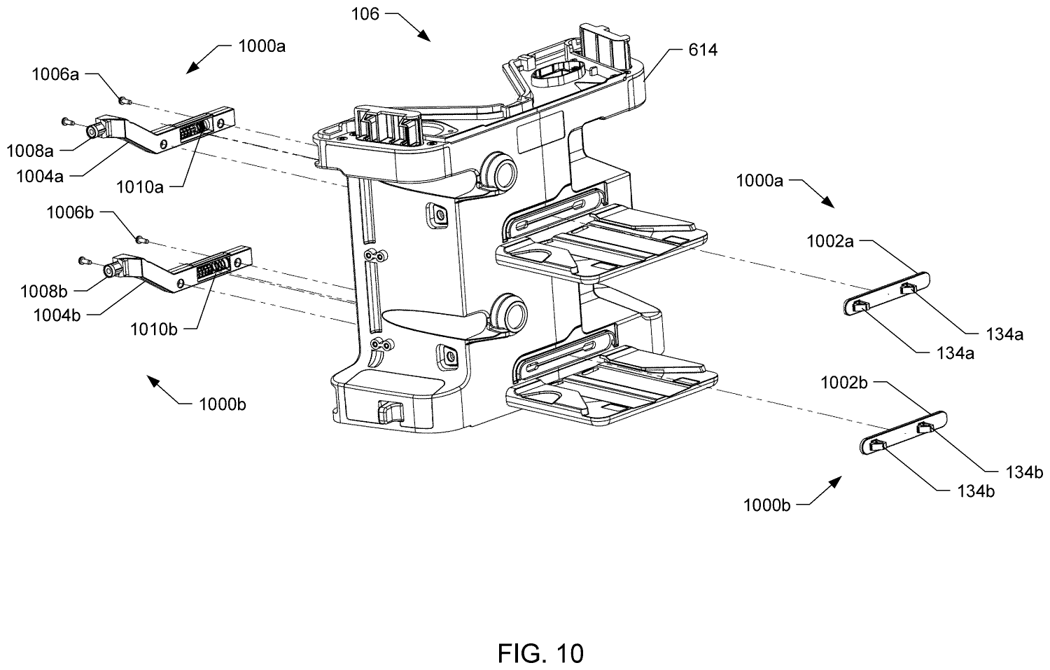

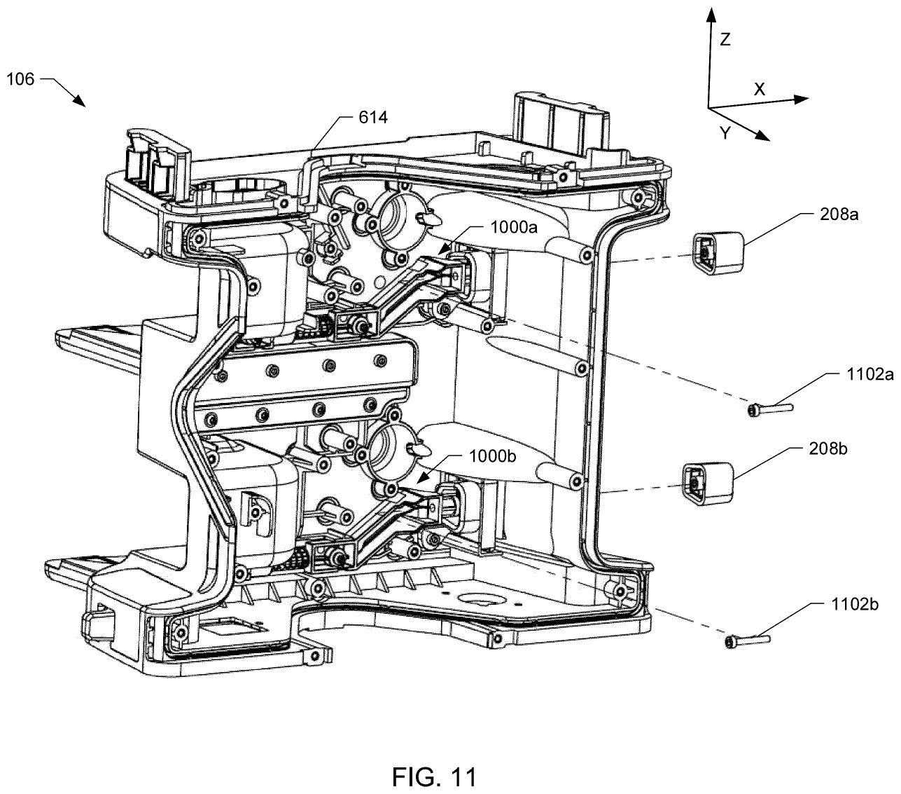

FIGS. 10 and 11 are diagrams illustrating components of medical device latching features 1000a, 1000b, that may be used with the docking apparatus 106 of FIGS. 1 to 3, according to an example embodiment of the present disclosure. FIG. 10 shows a first exploded view of the medical device latching features 1000a, 1000b from a front perspective of the docking apparatus 106. FIG. 11 shows a second exploded view of the medical device latching features 1000a, 1000b, from a rear view.

Each of the example medical device latching features 1000a, 1000b are configured to releasably couple a medical device to the docking apparatus 106. The example medical device latching feature 1000a includes a front portion 1002a including two of the latches 134a. The example front portion 1002a is installed on an exterior surface of the housing 614 of the docking apparatus 106. The example latches 134a are configured to mate with a recess section of a medical device. For example, the latches 134a may include a tab or other mechanical connector that engages a corresponding recess section of a medical device and slots or protrusions within the recess section of the medical device. Insertion of the latches 143a into the recess section may create a secure connection between the docking apparatus 106 and the medical device when the latches 134a are engaged. However, when the latches 134a are disengaged, the latches 134a may be slid from the recess section of the medical device, thereby enabling separation of the medical device from the docking apparatus 106.

The example medical device latching feature 1000a further includes a mechanism portion 1004a. The example mechanism portion 1004a is installed on an interior surface of the housing 614 of the docking apparatus 106. The example mechanism portion 1004 is coupled to the front portion 1002a via mounting hardware 1006a. Exterior of a side surface of the housing 614, the medical device release button 208a is coupled to a button protrusion 1008a of the mechanism portion 1004a via mounting hardware 1102b.

When no force is applied to the example medical device release button 208a, medical device latching feature 1000a including the example latches 134a remains in the engaged state. For example, with no force applied to the medical device release button 208a, the latches 134a may be engaged with a recess section of a medical device creating a secure connection between the docking apparatus 106 and the medical device. The medical device release button 208a may be actuated by applying a force to the medical device release button 208a in the negative x-direction. When the medical device release button 208a is engaged, the medical device latching feature 1000a including the latches 134a is moved to a disengaged position. For example, when the latches 134a are moved to the disengaged position, the latches 134a may no longer engage the recess section of the medical device. Thus, the medical device is disengaged from the docking apparatus 106 and may be removed.

The mechanism portion 1004a further includes a spring 1010a. The example spring 1010a may be a compression spring such as a coil compression spring. The example spring 1010a is configured to return the medical device latching feature 1000a to the engaged position after the medical device release button is engaged 208a.

The example medical device latching feature 1000a may further include a gasket (not pictured). The example gasket may be positioned between the button protrusion 1008b and the medical device release button 208b in order to prevent debris or fluid ingress into the housing 614 of the docking apparatus. The example gasket may be flexible to allow for travel of the medical device release button 208b while still preventing ingress of debris and fluid.

The example docking apparatus 106 further includes medical device latching feature 1000b having front portion 1002b, mechanism portion 1004b, mounting hardware 1006b, button protrusion 1008b, spring 1010b, and medical device release button 208b which may be substantially the same as the respective components of medical device latching feature 1000a. While the example of FIGS. 10 and 11 illustrates a docking apparatus 106 with two medical device latching features 1000a, 1000b, an example docking apparatus may include only one medical device latching assembly or more than two (e.g., three, four, etc.) medical device latching assemblies.

### CONCLUSION

It should be understood that various changes and modifications to the example embodiments described herein will be apparent to those skilled in the art. Such changes and modifications can be made without departing from the spirit and scope of the present subject matter and without diminishing its intended advantages. It is therefore intended that such changes and modifications be covered by the appended claims.

It should be appreciated that 35 U.S.C. 112(f) or pre-AIA 35 U.S.C 112, paragraph 6 is not intended to be invoked unless the terms “means” or “step” are explicitly recited in the claims. Accordingly, the claims are not meant to be limited to the corresponding structure, material, or actions described in the specification or equivalents thereof.

The invention is claimed as follows:

1. An infusion pump docking apparatus of a hub connectivity station, the docking apparatus comprising:
a housing; and
a device connector assembly comprising:
a mounting bracket fixedly connected to an interior surface of the housing forming a mounting cavity between the mounting bracket and the housing,
a device connector to provide electrical connection between the docking apparatus and a medical device, the device connector positioned in the mounting cavity, and
a spring positioned in the mounting cavity between a flange surface of the mounting bracket and a rear surface of the device connector, the spring deformable during connection of the medical device to the device connector to allow movement of the device connector within the mounting cavity.

2. The apparatus of claim 1, wherein the device connector assembly further comprises a front seal around the device connector, the front seal proximate an exterior surface of the housing.

3. The apparatus of claim 1, wherein the medical device is an infusion pump.

4. The apparatus of claim 1, wherein the device connector has freedom of motion in one or more directions.

5. The apparatus of claim 1, wherein the device connector moves from an initial position upon mounting of the medical device to the docking apparatus.

6. The apparatus of claim 5, wherein the interior surface of the housing proximate the device connector includes a housing tapered surface and the device connector includes a connector tapered surface.

7. The apparatus of claim 6, wherein the housing tapered surface and the connector tapered surface interact such that the device connector returns to the initial position after dismount of the medical device.

8. The apparatus of claim 1, wherein the device connector provides one or more of a controller area network connection or an Ethernet connection.

9. The apparatus of claim 1, wherein the device connector provides DC power to the medical device.

10. The apparatus of claim 1, wherein the device connector provides data routing between the medical device and the docking apparatus.

11. The apparatus of claim 1, wherein the device connector assembly further includes one or more wires extending from the device connector.

12. The apparatus of claim 1, wherein the device connector further includes a cylindrical protrusion extending from an exterior surface of the housing and a base extending from an interior surface of the housing.

13. The apparatus of claim 12, wherein the cylindrical protrusion of the device connector further includes a connection cavity extending inward from a front surface of the cylindrical protrusion.

14. The apparatus of claim 13, wherein the device connector further includes one or more pins extending outward from a rear surface of the connection cavity.

15. The apparatus of claim 14, wherein the movement of the device connector within the mounting cavity during connection of the medical device guides a connector of the medical device to the one or more pins to form the electrical connection.

16. An infusion pump docking apparatus of a hub connectivity station, the docking apparatus comprising:
a housing;
a first device connector assembly comprising:
a first mounting bracket fixedly connected to an interior surface of the housing forming a first mounting cavity between the first mounting bracket and the housing,
a first device connector to provide electrical connection between the docking apparatus and a first medical device, the first device connector positioned in the first mounting cavity, and
a first spring positioned in the first mounting cavity between a flange surface of the first mounting bracket and a rear surface of the first device connector; and
a second device connector assembly comprising:
a second mounting bracket fixedly connected to an interior surface of the housing forming a second mounting cavity between the second mounting bracket and the housing,
a second device connector to provide electrical connection between the docking apparatus and a second medical device, the second device connector positioned in the second mounting cavity, and
a second spring positioned in the second mounting cavity between a flange surface of the second mounting bracket and a rear surface of the second device connector.

17. The apparatus of claim 16, wherein the first medical device is a first type of medical device and the second medical device is a second type of medical device.

18. An infusion pump docking apparatus of a hub connectivity station, the docking apparatus comprising:
a housing;
a device connector assembly comprising:
a mounting bracket fixedly connected to an interior surface of the housing forming a mounting cavity between the mounting bracket and the housing,
a device connector to provide electrical connection between the docking apparatus and a medical device, the device connector positioned in the mounting cavity, and
a spring positioned in the mounting cavity between a flange surface of the mounting bracket and a rear surface of the device connector;
one or more couplings to provide mechanical coupling between the housing and the medical device; and
a shelf extending from the housing, the shelf to support a weight of the medical device when connected to the housing.

19. The apparatus of claim 18, wherein the shelf includes one or more recessed sections to guide the medical device into the one or more couplings.

20. The apparatus of claim 18, further including a release mechanism to decouple the one or more couplings from the medical devices.
